(19) United States
(12) Patent Application Publication
(10) Pub. No.: US 2004/0247665 A1
(43) Pub. Date: Dec. 9, 2004

(54) Pill carrier and method

(76) Inventors: Roy Smith, Naples, FL (US); Kenneth J. Smith, Naples, FL (US); Ralph Palermo, Naples, FL (US)

Correspondence Address: Michael A. Slavin, McHale & Slavin, P.A., Suite 402, 4440 PGA Boulevard, Palm Beach Gardens, FL, 33410, US

(21) Appl. No.: 10/457,731

(22) Filed: Jun. 5, 2003

(51) Int. Cl. A61K 9/20; B65D 83/04; B65D 85/42

(52) U.S. Cl. 424/465; 206/539

(57) ABSTRACT

A device for extruding a carrier for enclosing pills, tablets, or capsules allowing for ease of oral consumption by the recipient. The device dispenses a material from a stored volume to a formed tubular chamber allowing for the insertion of medication therein. The carrier can then be deformable to enclose and secure the medication within the chamber. The carrier may be used to mask the scent of medication and further provide a lubricant to allow for ease of consumption. The carrier is preferably formed from a composition of wheat flour, water, natural vegetarian based beef and mesquite flavors, preservatives, lubricants, with the remainder of the materials being inert.

### FIELD OF THE INVENTION

This invention relates generally to the medication of humans and animals, specifically, to a package that extrudes a formed carrier having a chamber for pills, tablets, or capsules. The carrier masks the scent of medication and includes a lubricant to allow for ease of consumption.

### BACKGROUND OF THE INVENTION

Infants, children and some adults find it difficult to swallow hard dry medications of certain types. In addition to recalcitrance, the problem may relate to fear, or the size of the medication, or the inability to quickly wet the medication reducing the resistence to swallowing.

Pet owners share a common problem in the dispensing of medication to their pets. This is a particular problem with a dog which is capable of detecting a scent over one hundred times greater than a human. The dog is able to detect medication which is found offensive to most pets. For this reason, it is difficult to cause the animal to swallow the medication.

With small dogs, a pill or tablet may be large enough that the animal must first chew the medication in order to swallow it. This may be performed by breaking up the medication, which can further release a strong medicine scent. Alternatively the medication may be forced down the dog's throat. In larger dogs, there is a risk that the physical insertion of such medication into the dog's throat may result in a dog bite.

For these reasons, various types of medicine applications have been attempted, some of which work well for some humans and some dogs yet fail with others. For example, it may be possible to mix the medication within food wherein the medication is consumed as a treat.

However, dog foods are natural foods that cannot mask the scent of normal medication. Typically the sprinkling of the medication on the dog food may cause the dog to reject eating, leading to further problems. Yet another method of inserting medication is the use of a piece of meat wherein the medication is pressed into the food and the food delivered to the animal in the form of a dog treat. In most instances, this application also fails as the owner handles the medication during the insertion process which in turn covers the outer surface of the food with the scent. In addition, the meat must be of the correct substance for if it is too dry, it will fall apart or cause the animal to choke. If the meat is too moist, it will be difficult to work with and will not hold the medication. Further, high moisture will cause the medication to dissolve which may cause the release of the medication scent. If the scent is released, the dog will again be able to detect the scent of the medication either through the direct access or through the handling of the food.

Various devices have been patented so as to assist in this pill dispensing process, the obvious discomfort to a small animal is readily apparent. U.S. Pat. No. 5,584,805 discloses a pill-dispensing gun made from PVC pipe having a core with a rigid tube which extends through the tube and then couples to a plunger. The PVC pipe is inserted down the animal's throat when a plunger injects the pills into the animal's throat in such a position so as to force swallowing of the pill.

U.S. Pat. No. 5,399,162 discloses a device that also is used for insertion of medication down the throat of the animal. A spring biased rod is used for injecting the medication down the animal's throat.

U.S. Pat. No. 3,789,845 discloses a syringe type holder used for administrating the capsules or tablets to the animals. This device uses a sticky food material to adhere a tablet to a plug which is then inserted down the animal's throat. When it is far enough, the syringe pressure is released allowing release of medication.

U.S. Pat. No. 5,853,757 discloses a composition carrier formed from soy flour with a lubricant. The composition maintains a level of moisture which facilitates closure and assists the recipient in swallowing of the carrier and associated medication.

Thus, what is lacking in the art is a device that provides a formed carrier for the inexpensive administration of medicine; the carrier being capable of masking the medication scent and includes a lubricant to assist the recipient in swallowing.

### SUMMARY OF THE INVENTION

The present invention consists of a package filled with a digestible carrier and composition that allows for ease of delivering a tablet, capsule, pill with like medication to a human or animal. The carrier is formed from a material that is found most favorable by humans and animals and forms such a chamber structure for securing medication.

The chamber is available for receipt of medication which formed as an extrusion wherein the medication can be sealed into the chamber upon the deformation of the carrier. Preferably the carrier masks the scent of the medication wherein the carrier can be swallowed whole, wherein a lubricant assists such an act, or chewed wherein the carrier material of construction is formed by edible formula's found favorable to most humans and animals. Of course, other suitable food products may be used, including all those edible products that can be extruded from a paste into a self-sustaining enclosure formed tube.

In the preferred embodiment, a carrier is formed from a composition of wheat flour, water, natural vegetarian based beef and mesquite flavors, preservatives, lubricants, with the remainder of the materials being inert. By manual manipulation of the device, the composition is formed into a tubular shape. The composition maintains a level of moisture which facilitates closure for sealing the medication in the chamber. The lubricant assists the recipient in swallowing of the carrier and associated medication. The lubricant reacts with the saliva to prevent the recipient from choking on the hard medicine.

Thus, an objective of the instant invention is to provide a simple and inexpensive package and carrier for delivery of medicine to animals without the need for insertion of objects into the throat.

Another object of the instant invention is to set forth a device for use in administering medicine that conceals the scent of the medication and minimizes handling.

Yet still another object of the instant invention is to form a chamber which allows for the insertion of various types of oral medication so as to seal the medication within a formed chamber to prohibit the recipient from seeing or smelling of the medication before the medication is placed into the recipient's mouth.

Still yet another object of the instant invention is to provide a carrier for medicine which allows the recipient to consume the medication with a passive positioning of the medication to inspire relaxation and calmness.

Yet still another object of the instant invention is to provide a carrier that operates as for concealment of liquid or solid medications.

Yet another object of the instant invention is to provide a carrier that is firm enough to maintain a shaped chamber yet includes a lubricant to assist the recipient in swallowing of the carrier.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

FIG. 1 is a cross section of the package of this invention;

FIG. 2 is a cross section of the mouth of the package;

FIG. 3 is a cross section of FIG. 2 along line 3-3;

FIG. 4 is a cross section of the body of the package;

FIG. 5 is a plan view of the plunger of the package;

FIG. 6 is a side view of the guide of the package;

FIG. 7 is a perspective of the carrier of this invention;

FIG. 8 is a perspective of the carrier of this invention.

### DETAILED DESCRIPTION OF THE PREFERRED EMBODIMENT

Referring to FIG. 1, the package 20 is shown as a container having a bottom 21 and a mouth 22 joined by a continuous side wall 23. The bottom 21 has a hub 24 extending into the interior space of the package with an external screw cap 25 sealed therein. As shown in FIGS. 4 and 5, the screw cap has a base 26 with at least one sealing bearing 27 closing the hub but allowing rotation of the screw cap. The hub may have a flange 28 cooperating with a groove 29 in the screw cap to provide a first rotary seal.

A screw jack 30, in FIG. 6, having external threads extends from the base 26. A plunger 31 is mounted on the screw jack 30 by internal threads 32 formed in an aperture 33. The plunger 31 has a wall 34 about the periphery with slots 35, 36 and 37 formed therein. The wall 34 acts on the sidewall 23 to prevent the plunger from tilting out of plane and jamming. The internal threads 32 and the screw jack threads cooperate to move the plunger 31 away from the base 26 by turning either the screw cap 25 or package 10 relative to the other. The sidewall 23 has longitudinal guide rails 38 and 39, shown, which slidingly fit within the slots in the plunger to restrict rotary movement of the plunger as it moves axially along the screw jack.

In FIGS. 2 and 3, the mouth 22 is shown with a sealing ring 40 for connection with the top end of the package 10. An occlusion 41 is suspended inside the mouth 40 by a web of supports 42, 43, 44, and 45. The occlusion 41 blocks the central portion of the mouth 22 leaving a ring-like opening 46 communicating with the interior space of the package. A re-closable protective cap (not shown) fits over the mouth and closes the package.

In operation, the plunger rests on the base of the screw jack and the interior space of the package is filled with an carrier material in paste form.

To administer a medication, the screw cap and package are rotated relative to each other causing the plunger to move axially toward the mouth of the package. The carrier is forced through the web and into the ring-like opening of the mouth and emerges as a formed tube of varying desired length.

Other modifications of the package 20 include a soft sided container that may be squeezed or rolled to express the carrier.

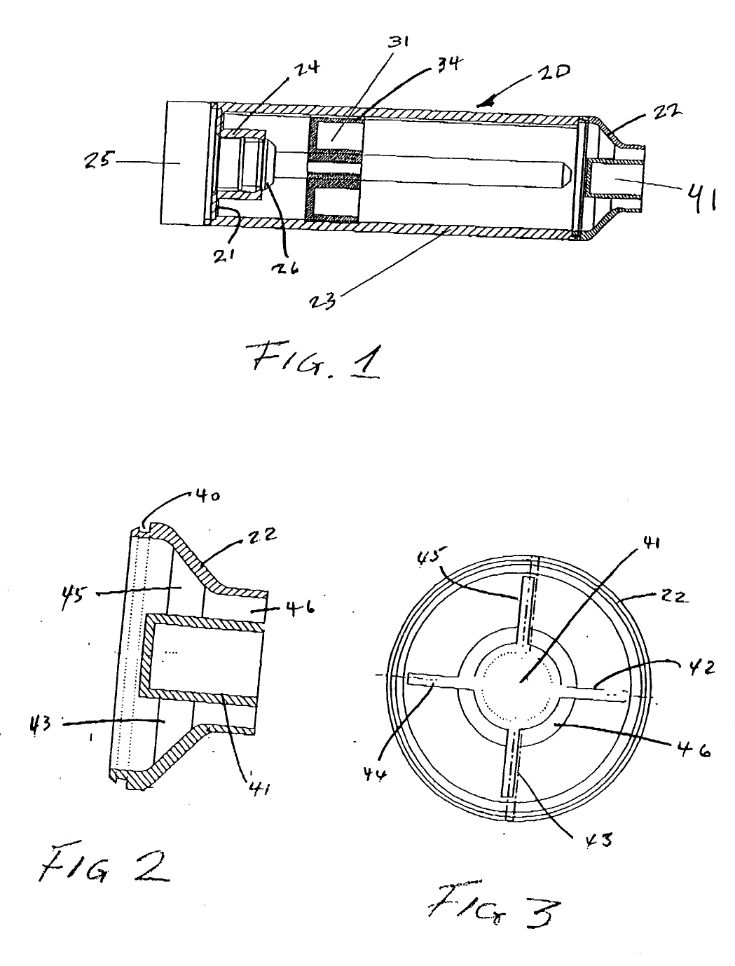

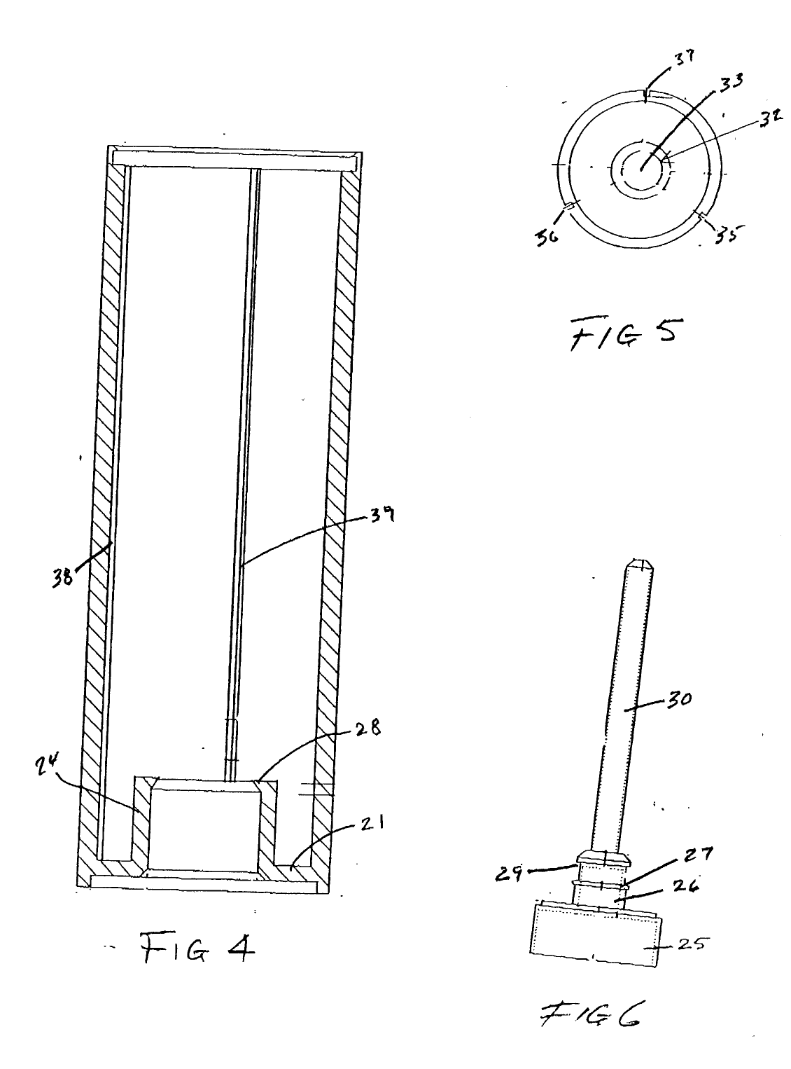

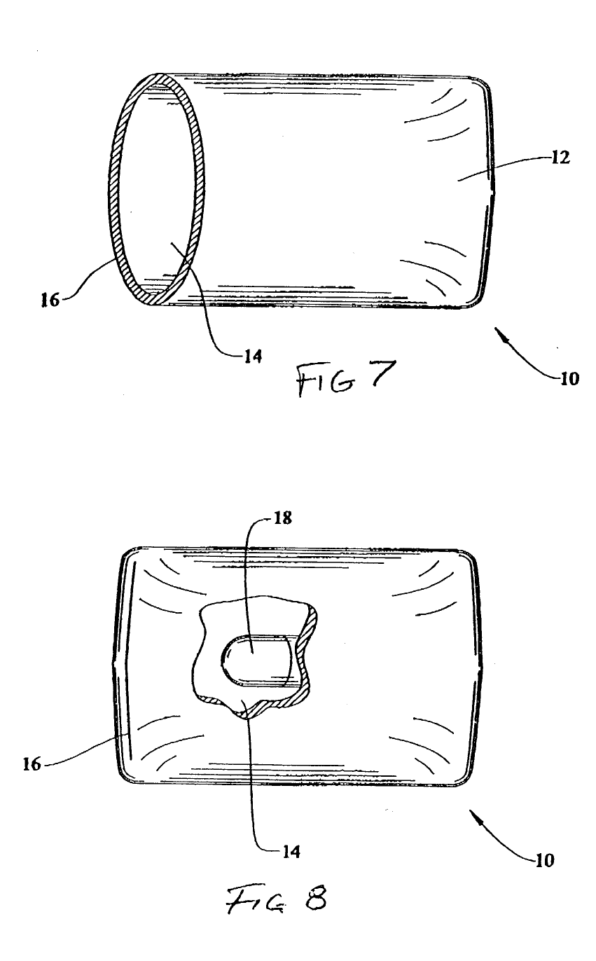

Referring to FIGS. 7 and 8, set forth is the carrier 10 of the instant invention for use with animal medication. The carrier is formed from soft edible material 12 with a tube-like structure and an interior chamber 14 accessible by a neck opening 16. The carrier 10 is of a size to contain a medicament 18 shown as a capsule.

The carrier is formed from a wheat flour, water, natural vegetarian based beef and mesquite, flours, preservative, lubricants, with the remainder of the materials being inert.

The flour or other thickening agent maintains the carrier in a soft pliable state when used with a water soluble lubricants. Should the carrier be swallowed as a whole, the lubricant reacts with the saliva causing a slick surface allowing the carrier to swallowed without discomfort to the animal. Other materials may be used as the lubricant including modified cellulosic products, gelatin, and other compounds that react with saliva to produce a slick surface.

Upon use, the package is manipulated to move the plunger along the guide which extrudes a tubular carrier from the mouth of the package. An individual may grasp one end of the tubular carrier in one hand to close one end of the tube. In operation, the user first inserts a pill into the opening 41, then extrudes formula to cover the pill, then pinches the formula closed around the pill, then extrudes more formula to cover the pill length, then removes extruded formula and pill from the device closing the formula around the pill. The neck opening allows for ease of insertion eliminating the need for having the hand touching the medication also touch the outside of the carrier. Thus, once the medication has been inserted, the individual squeezes the carrier shut to seal the medication within the chamber. By use of one hand hold the carrier, the opposite hand can be used to insert the medication. The carrier is then closed by the hand used to hold the carrier to avoid placing the scent on the outside of the carrier. The carrier can then be placed into the mouth of the recipient wherein the recipient will either swallow the carrier whole, or chew the carrier wherein the formula masks the flavor of the medicine.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown in the drawings and described in the specification.

What is claimed is:

1. A method of making an individual medication carrier comprising the steps of;
providing an carrier package, said package having an enclosed internal space, an carrier in paste form located in said space, said package having a mouth, said mouth having an occlusion with a concentric opening,
manipulating said package to reduce said internal space and force said carrier through said mouth, said mouth forming said carrier into a tubular form; and
removing said tubular form from said mouth.

2. An individual carrier made by the method of claim 1 wherein said formed tube comprises an edible food product including a thickening agent and a lubricant, said formed tube of a size to enclose a medication.

3. An individual carrier made by the method of claim 1 wherein said mouth is sized to receive a medicate.

4. A method of forming a combination medication and carrier comprising the method of making a medication carrier of claim 1;
and comprising the further steps of;
forming a combination medication and carrier by placing a medication within said formed tube;
sealing said combination medication and carrier by closing said formed tube about said medication; and
administering said combination to a recipient.

5. A combination carrier and medication including the steps of claim 4;
wherein said formed tube comprises an edible food product including a thickening agent and a lubricant, said formed tube of a size to enclose a medication.

6. A method of making an individual medication carrier of claim 1 comprising the further steps of;
providing a axially movable plunger in said interior space with said carrier disposed between said plunger and said mouth; and
manually manipulating said package to move said plunger toward said mouth reducing said internal space and forcing said carrier through said mouth.

7. A method of making an individual medication carrier of claim 6 comprising the further steps of;
providing guide rails in said internal space of said package, said plunger axially movable along said guide rails;
providing said plunger with a threaded aperture therethrough; and
providing said package with a closed bottom opposite said mouth, said bottom having a screw cap with a threaded screw jack extending into said internal space, said plunger threaded onto said screw jack for axial movement upon rotation of said screw cap.

8. A method of administering medication of claim 4 comprising the further steps of providing said medication in the form of one of the group consisting of a pill, capsule, tablet.

9. A method of administering medication of claim 6 comprising the further steps of positioning said medication in said mouth.

10. A method of forming a combination medication and carrier comprising the method of making a medication carrier of claim 1;
and comprising the further steps of forming a combination medication and carrier by:
placing a medication in said mouth;
extruding said paste into a formed tube around said medication;
sealing said medication by closing said formed tube about said medication; and
administering said combination to a recipient.

11. An carrier for administering medication comprising a soft edible form having a tubular chamber with open ends, said carrier having a lubricant admixed in said formed tube wherein said chamber is adapted to accept a medication and said open ends are sealable in a closed position for securing the medication in said chamber.

12. The carrier for animal medication according to claim 11 wherein said carrier is a composition comprising wheat flour, water, natural vegetarian based beef and mesquite flavors, preservatives, and a lubricant.

13. A medication carrier comprising;
an carrier package, said package having an enclosed internal space, an carrier in paste form located in said space, said package having a mouth, said mouth having an occlusion with a concentric opening;
said tubular paste comprises an edible food product including a thickening agent and a lubricant, said paste being a dough-like consistency;
an axially movable plunger in said interior space with said carrier disposed between said plunger and said mouth;
guide rails in said internal space of said package, said plunger axially movable along said guide rails;
said plunger with a threaded aperture therethrough;
said package having a closed bottom opposite said mouth, said bottom having a screw cap with a threaded screw jack extending into said internal space, said plunger threaded onto said screw jack for axial movement upon rotation of said screw cap;
whereby manipulating said package reduces said internal space and forces said carrier through said mouth, said mouth forming said carrier into a tube.

14. The paste according to claim 13 wherein said paste is formed from a composition of wheat flour, water, natural vegetarian based beef flavors and preservatives.

15. The paste according to claim 13 wherein said paste is formed from a composition of wheat flour, water, natural vegetarian based mesquite flavors and preservatives.
